(19) United States
(12) Patent Application Publication
(10) Pub. No.: US 2024/0188650 A1
(43) Pub. Date: Jun. 13, 2024

(54) DEVICE AND METHOD FOR SUPPORTING AND DISTRIBUTING WEIGHT OF THE ABDOMEN OF A PERSON

(71) Applicant: HUGUP SP. ZOO, Poznan (PL)

(72) Inventors: Slawa Helena MADELSKA, Poznan (PL); Matylda Teresa CIANCIARA, Poznan (PL); Weronika Julia DRAUS, Warsaw (PL); Kamila Olga PRZYBYLSKA, Poznan (PL)

(21) Appl. No.: 18/538,348

(22) Filed: Dec. 13, 2023

(30) Foreign Application Priority Data
Dec. 13, 2022 (CA) 3184003

(51) Int. Cl. A41C 1/10; A41D 1/21

(52) U.S. Cl. CPC A41C 1/10; A41D 1/21; A41D 2400/38; A41D 2500/10

(57) ABSTRACT

A body sling to support the weight of a growing fetus and redistribute the load onto the shoulders and trunk is provided. The body sling comprises a plurality of functional regions designed for optimal support and comfort. The body sling 100 comprises a series of elastic sections made of a material providing adaptable tension and accommodating the changing contours of a pregnant wearer's body. The sling further comprises non-elastic sections interspersed with the elastic sections. The non-elastic sections are generally and strategically integrated to the sling to provide consistent support and to ensure that the sling retains its structural integrity while in use.

### CROSS-REFERENCE TO RELATED APPLICATIONS

This application claims priority to Canadian patent application number 3,184,003, filed on Dec. 13, 2022, and titled “Device and method for supporting and distributing weight of the abdomen of a person”, the entire contents of which are incorporated herein by reference.

### FIELD OF THE INVENTION

The present invention generally relates to devices and methods for supporting and distributing weight of the abdomen of a person. More particularly, the present invention relates to methods and garments designed to support the abdomen of a pregnant woman and, more particularly to garments designed to alleviate pain and discomfort by redistributing load onto the entire trunk, as well as reducing the pressure on organs within the lumbar and pelvic area.

### BACKGROUND OF THE INVENTION

During and after pregnancy, joint laxity and swelling of the abdomen are associated with fetal growth, which changes the pelvic alignment of women. For example, softening of the ligaments that join the pelvis occur because of pregnancy-related hormones such as relaxing which function is to make space for fetal growth. In addition, the pelvis widens as the abdomen enlarges with the advancing pregnancy and to facilitate the forthcoming labor and delivery. Although widening of the pelvis is necessary for fetal growth and childbirth, significant problems such as separation of the pubic symphysis can occur when pelvic widening exceeds the limits of its range of motion and pelvic joint flexibility. Pelvic asymmetry alters the body's mechanics, placing strain on various body segments, which subsequently contributes to musculoskeletal pain.

Research also suggests that changes in pelvic alignment that occur in pregnancy might persist after pregnancy. As such, pelvic belts are popular among these women as a tool to prevent changes in pelvic alignment in addition to reducing lumbopelvic pain. Devices that help to stabilize the pelvis such as pelvic belts are thought to be effective in relieving lumbar and pelvic pain.

It has been suggested that hip adduction forces in patients with pregnancy-related pelvic girdle pain is increased, the intensity of pelvic pain is reduced, and that daily activities are improved with pregnancy belt use. These effects are thought to occur because the belts, which compress the pelvis externally, are thought to augment pelvic stability via additional closure forces in lumbopelvic disorders where stability is compromised. This function of the pelvic belt might affect the width of the pelvis. Moreover, this pelvic compression function inhibits excessive movement of the pelvis joint and might also correct pelvic asymmetry.

However, the existing pregnancy belts on the market do not provide an optimal weight distribution, most often focusing only on the lumbar region, which significantly burdens the area already extremely vulnerable to pain during pregnancy. Furthermore, the lack of adjustability of these belts leads to discomfort for the expecting mother as well as quickly becomes inadequate as the baby grows and load increases. Moreover, users of existing pelvic belts and garments often complain of poor breathability of the fabric and lack of moisture wicking capability. Lastly, ergonomic considerations both for putting on and off and for adjustments are often lacking in these devices.

There is thus a need for a novel device for supporting and distributing weight during maternity and for a method for doing the same.

### SUMMARY OF THE INVENTION

The shortcomings of the prior art are generally mitigated by an adjustable and easily wearable maternity sling which provides optimal weight distribution.

This maternity body support is worn to support the weight of a growing fetus and redistribute the load onto the shoulders and trunk of a women during maternity. The body support generally comprises a continuous sling with built-in fastening points. The body support may be manufactured through 3D knitting allowing variability in the sling's properties. The change in material properties and the construction of the angles and the anatomical fit of the individual parts of the garment design allows for increased wearing comfort and improved performance. Moreover, fastening points allow both adjustment of the overall length and tension of the system, also allowing the user to rapidly and easily put it on and off.

In one aspect of the invention, a continuous body sling for supporting and distributing the load of the abdomen of a wearer is provided. The body sling comprises a pelvic portion positioned to support the belly of the wearer and to stabilize the pelvis of the wearer, a shoulder portion adapted to wrap around the shoulders of the wearer to transfer at least some of the load onto the shoulders and neck, at least one crossing of the sling in the back of the wearer to distribute some of the load onto the trunk and a fastening mechanism adapted to vary the length of the worn sling at a plurality of fastening points, wherein at least one of the portions is made of elastic material to accommodate changing contours of a wearer's body.

The pelvic portion may be anatomically shaped to fit the body of the wearer and may be positioned at a first position in the suprapubic or hypogastric region supporting the weight of the belly of a wearer.

The body sling may form two or three crossings in the back of the wearer and may be made of 3D knitted fabric, of moisture wicking and breathable material, of heat transferring material and/or with material alternating between material having different elastic properties. The material may alternate between elastic and non-elastic material. The material may alternate between elastic and semi-rigid material.

The series of elastic sections provided in the sling may be interspersed with non-elastic sections, wherein the non-elastic sections are integrated to furnish consistent support, ensuring that the sling retains its structural integrity while in use.

The material may have higher rigidity in the pelvic portion. The material of the sling may be more supple in portions of the sling to be worn on the axillary region of the wearer than in other portions of the sling.

The fastening mechanism may comprise a detachable buckle allowing adjustment of strap length to personal setting. The detachable buckle comprising multiple tension settings.

The body sling may comprise multiple shoulder portions to improve load distribution. The body sling may be adaptable to the weight of the abdomen being increased by a growing foetus, wherein the wearer is a pregnant woman. The weight of the abdomen may be increased by a growing foetus and the wearer may be a pregnant woman.

In another aspect of the invention, a bodysuit comprising tunnels for allowing passage of a built-in body sling for supporting and distributing the load of the abdomen of a wearer throughout the body trunk of the said wearer is provided. The body sling comprises shoulder portions to transfer at least some of the load onto the shoulders and neck, a pelvic portion positioned between the shoulder portions to support the belly of the wearer and stabilizing the pelvis of the wearer and at least one crossing of the sling in the back of the wearer to distribute some of the load onto the trunk of the wearer, wherein at least one of the portions of the body sling is made of elastic material to accommodate changing contours of a wearer's body.

The bodysuit may be made of moisture wicking, heat transferring and breathable material and/or may be 3D knitted. The built-in body sling may form two or three crossings in the back of the wearer.

The series of elastic sections of the sling may be interspersed with non-elastic sections, wherein the non-elastic sections are integrated to furnish consistent support, ensuring that the sling retains its structural integrity while in use. The bodysuit may be maternity bodysuit. The bodysuit may be adaptable to the weight of the abdomen being increased by a growing foetus, wherein the wearer is a pregnant woman.

In yet another aspect of the invention, a method to support and distribute weight of the abdomen of a wearer throughout the body trunk of the said wearer is provided. The method comprises supporting a pelvic portion of the belly of the wearer using a continuous sling, wrapping the continuous sling around the shoulder and the trunk of the wearer, crossing the continuous sling in the back of wearer and attaching a first end of the sling to a second end of the sling to provide a sufficient compression level on the wearer.

The method may further comprise crossing the continuous sling a second time in the back of the wearer.

Other and further aspects and advantages of the present invention will be obvious upon an understanding of the illustrative embodiments about to be described or will be indicated in the appended claims, and various advantages not referred to herein will occur to one skilled in the art upon employment of the invention in practice.

### BRIEF DESCRIPTION OF THE DRAWINGS

The above and other aspects, features and advantages of the invention will become more readily apparent from the following description, reference being made to the accompanying drawings in which:

FIG. 1 is top plan view of an embodiment of a maternity body sling shown laid flat in accordance with the principles of the present invention.

FIG. 2 is an isometric view of a front lower portion of the maternity body sling of FIG. 1 shown being worn by a user.

FIG. 3 is a rear view of the lower portion of the body sling of FIG. 1, shown worn in first criss-cross back pattern.

FIG. 4 is a side view of the sling of FIG. 1 shown being donned on a user for enhanced stability and support.

FIG. 5 is a lateral perspective view of the sling of FIG. 1, shown being donned by a user.

FIG. 6 is a back view of the maternity body sling of FIG. 1 showing the second criss-cross back pattern.

FIG. 7 is a front view of the maternity body sling of FIG. 1 shown assembled before the size adjustment.

FIG. 8 is a side view of the maternity body sling showing a method for adjusting the size of the said maternity body sling.

FIG. 9 is a perspective view illustrating an embodiment of an assembling procedure of the body sling of FIG. 1, including size adjustment and arm regulation.

FIG. 10 is a back view of the maternity body sling of FIG. 1 showing a third criss-cross back pattern.

FIG. 11 is a side view of the maternity sling of FIG. 1 shown with one potential fastening point.

FIG. 12 is a perspective view of the maternity sling of FIG. 1, showing an embodiment of an adjustment mechanism utilising a fastening buckle.

FIG. 13 is a front view of the maternity sling of FIG. 1 shown fully adjusted to the user.

FIG. 14 is a back view of the maternity sling of FIG. 1 shown fully adjusted to the user.

FIG. 15 is a side view of an embodiment of a maternity body sling showing one of potential adjustable attachment points.

FIG. 16 is a side view of an embodiment of a maternity body sling showing alternative adjustable attachment point.

FIG. 17 is a top perspective view of an embodiment of a first portion of an elongated buckle to be used with a maternity body sling in accordance with the principles of the present invention.

FIG. 18 is a bottom perspective of a second portion of the elongated buckle of FIG. 17.

FIG. 19 presents a top perspective view of an embodiment of a first portion of a round rotational buckle to be used with a maternity body sling in accordance with the principles of the present invention.

FIG. 20 presents a bottom view of a second portion of the round rotational buckle of FIG. 19.

FIG. 21 is a side view of a pregnant women showing mechanical forces on the body without the maternity body sling.

FIG. 22 is a side view of a pregnant women wearing an embodiment of the maternity body sling showing mechanical forces on the body exerted by the maternity body sling.

FIG. 23 is a front view of a body mapping image showing the yarn weaving selection based on functionality.

FIG. 24 is a back view of a body mapping image showing the yarn weaving selection based on functionality.

FIG. 25 is a front view of an embodiment of a full-body garment in accordance with the principles of the present invention shown with tunnels where a sling is integrated into the full-body garment.

FIG. 26 is a back perspective view of an embodiment of the full-body of FIG. 25 shown with tunnels where the sling is integrated into the said full-body garment.

FIG. 27 is a front view of an embodiment of a complete body garment in accordance with the principles of the present invention shown without a sling.

FIG. 28 is a back perspective view of the complete body garment of FIG. 27 shown without a sling.

FIG. 29 is a front view of another embodiment of a complete body garment in accordance with the principles of the present invention, comprising various knitted patterns and shown before being integrated with a maternity sling.

FIG. 30 is a side view of the complete body garment of FIG. 29.

FIG. 31 is a back view of the complete body garment of FIG. 29.

FIG. 32 is a front view of yet another embodiment of a complete body garment in accordance with the principles of the present invention shown with an integrated, adjusted and fastened body sling.

FIG. 33 is a side view of an embodiment of the complete body garment of FIG. 32.

FIG. 34 is a back view of the complete body garment of FIG. 32.

FIG. 35 is a side view of an alternative embodiment of a maternity bodysuit with an underwear portion and integrated sling in accordance with the principles of the present invention.

FIG. 36 is a front view of the maternity bodysuit of FIG. 35.

FIG. 37 is a back view of the maternity bodysuit of FIG. 35.

FIG. 38 is a front view of another alternative embodiment of a maternity bodysuit with a bust supporting portion, an underwear portion and an integrated sling in accordance with the principles of the present invention.

FIG. 39 is a front view of another alternative embodiment of a maternity bodysuit with an underwear portion and an integrated sling in accordance with the principles of the present invention.

FIG. 40 is a back view of the maternity bodysuit of FIG. 39.

FIG. 41 is a front view of yet another embodiment of a maternity bodysuit with an underwear portion and integrated sling in accordance with the principles of the present invention.

FIG. 42 is a front view of a further alternative embodiment of a maternity bodysuit with an underwear portion and integrated sling being attached in accordance with the principles of the present invention.

FIG. 43 is a back view of the maternity bodysuit of FIG. 42.

FIG. 44 is a front view of an embodiment of a postpartum and general-use back support garment in accordance with the principles of the present invention.

FIG. 45 is a back view of the back support garment of FIG. 44.

FIG. 46 is a front view of another embodiment of a postpartum and general-use back support garment in accordance with the principles of the present invention.

FIG. 49 is a back view of the back support garment of FIG. 48.

### DETAILED DESCRIPTION OF THE INVENTION

A novel device and method for supporting and distributing weight of the abdomen of a person will be described hereinafter. Although the invention is described in terms of specific illustrative embodiments, it is to be understood that the embodiments described herein are by way of example only and that the scope of the invention is not intended to be limited thereby.

While the present description refers to an exemplary embodiment of a sling and/or a garment for a pregnant woman, the present invention may be used to support any other type of weight or load of the abdomen of a wearer, such as but not limited to a person being overweight or a person having a disease which increases the weight of the said person. Understandably, the presently disclosed device may be used to adapt to variations of the load of the abdomen of a wearer.

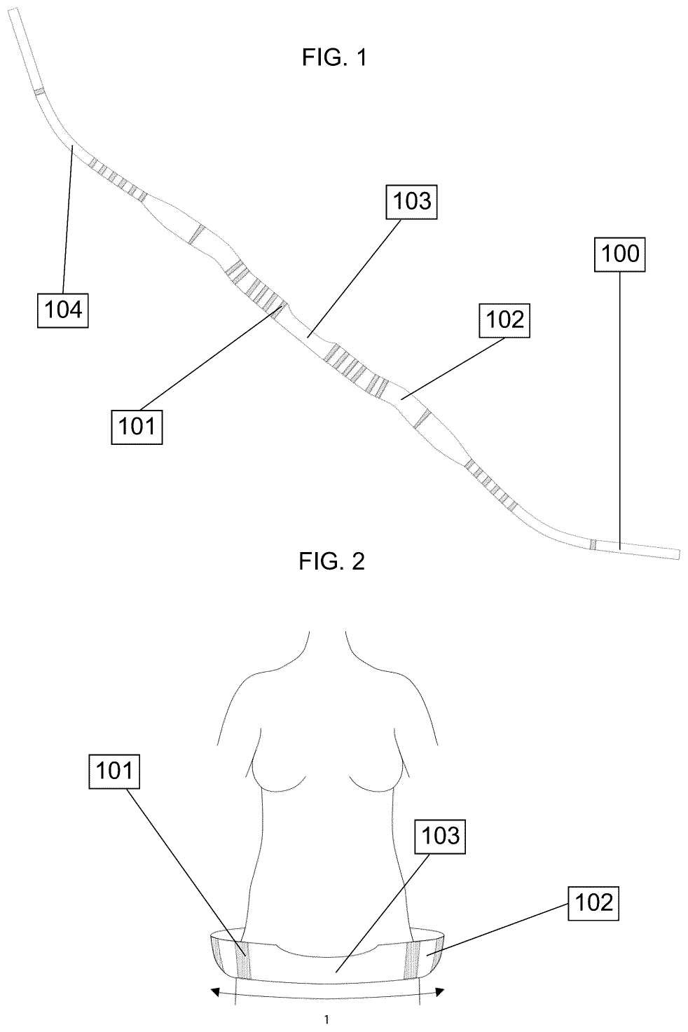

Referring now to FIG. 1, an exemplary embodiment of a maternity body sling as laid flat 100 is illustrated. The sling 100 comprises a plurality of functional regions designed for optimal support and comfort. In the illustrated embodiment, the body sling 100 comprises a series of elastic sections 101. The elastic sections 101 are made of a material providing adaptable tension and accommodating the changing contours of a pregnant wearer's body. The sling 100 further comprises non-elastic sections 102. The non-elastic sections 102 are interspersed with the elastic sections 101. The non-elastic sections 102 are generally and strategically integrated to the sling 100, generally aiming at furnishing consistent support and at ensuring that the sling retains its structural integrity while in use. In the illustrated embodiment, the sling comprises multiple curvatures and material variances generally resulting in an interplay of flexibility. The sling 100 further aims at providing close-fit and support.

The rigid or non-elastic portions 102 generally aim at supporting and stabilizing the pelvic girdle from below and upward. The elastic panels 101 are typically seamlessly inserted in the belly support sling 100 to allow the sling 100 to fit fully around the abdomen of a user and to better disperse or distribute weight by transferring the said weight to other parts of the body not involved in belly carrying.

The elastic panels 101 may further provide a smoothing effect. The elasticity in the thoracic spine generally promotes better adjustment and comfort for the user, while the support provided by rigid material panels aims at actively reduce kyphosis. The unique shape of the lower panel 103 adapts seamlessly to the lower abdominal shape and allows for stabilisation and improved posture in the pelvic area and hip spines. Additionally, the sling 100 further comprises one or more curves 104. The curve 104 is ergonomically-shaped to the user. The curve 104 is generally located in portions of the sling 100 that wrap around the shoulders of the wearer. The said curves 104 generally ensure an even more effective fit to the body and thus a better support and distribution of the abdominal weight.

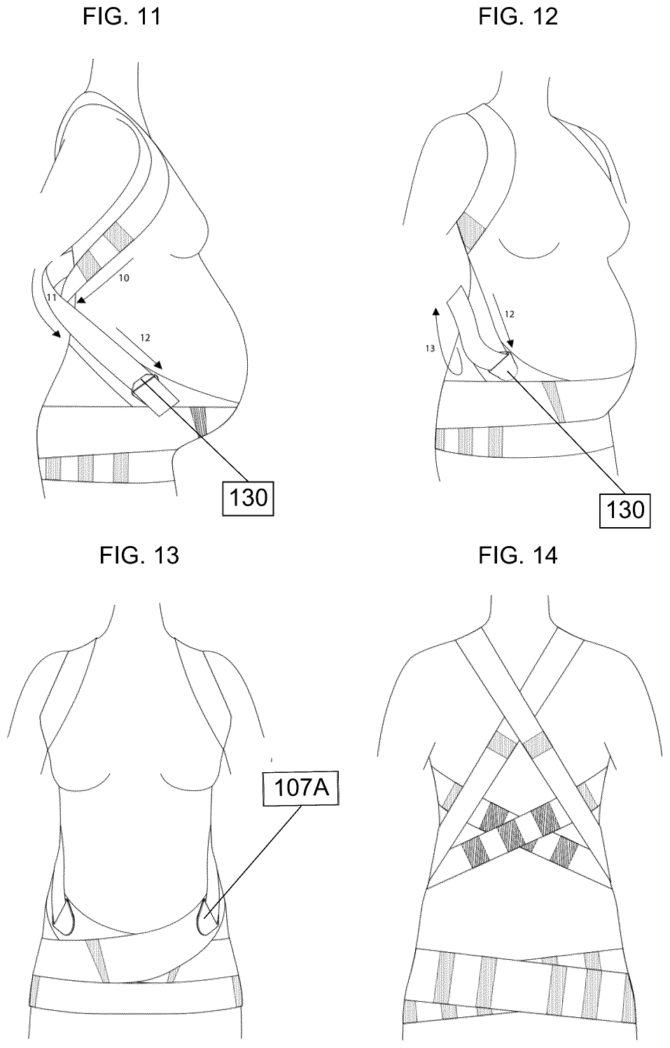

Referring to FIGS. 2 to 14, a garment for supporting and distributing weight 100 embodied as a string is illustrated. Each figure provides a sequential guide, solely to demonstrate an embodiment of the installation process in a preferred ordered manner. The sling string 100 comprises a wider and stiffer pelvic portion 103 (see FIG. 2). The pelvic portion is 103 generally at the center or mid-point of the sling 100. The pelvic portion 103 is generally anatomically shaped to fit the body and generally positioned at a first position in the suprapubic or hypogastric region supporting the weight of the belly of the pregnant wearer.

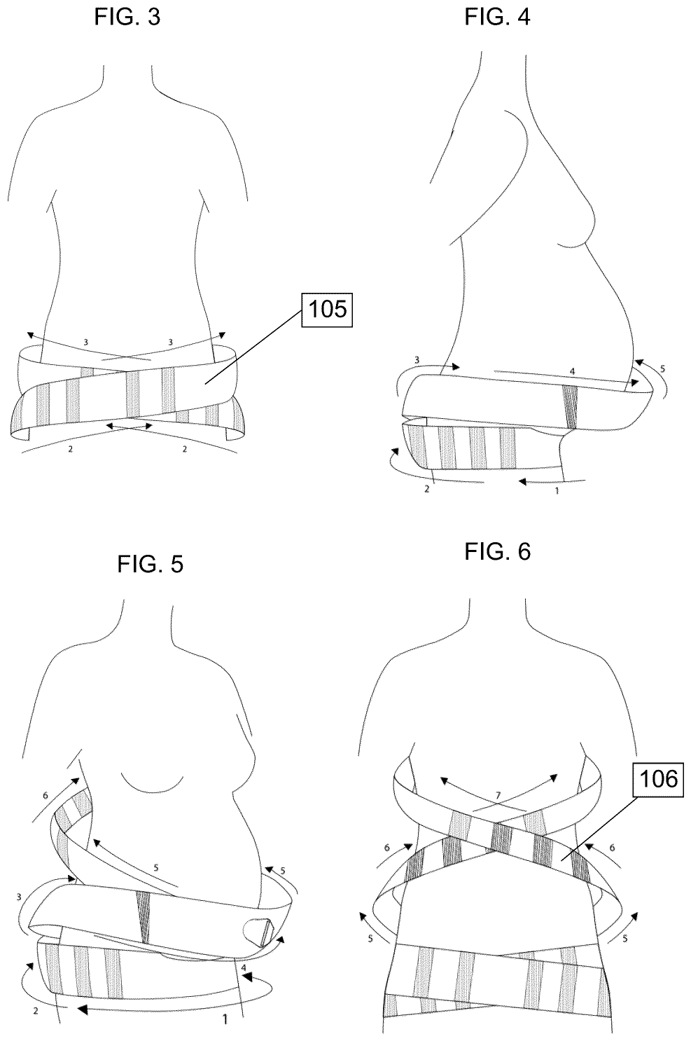

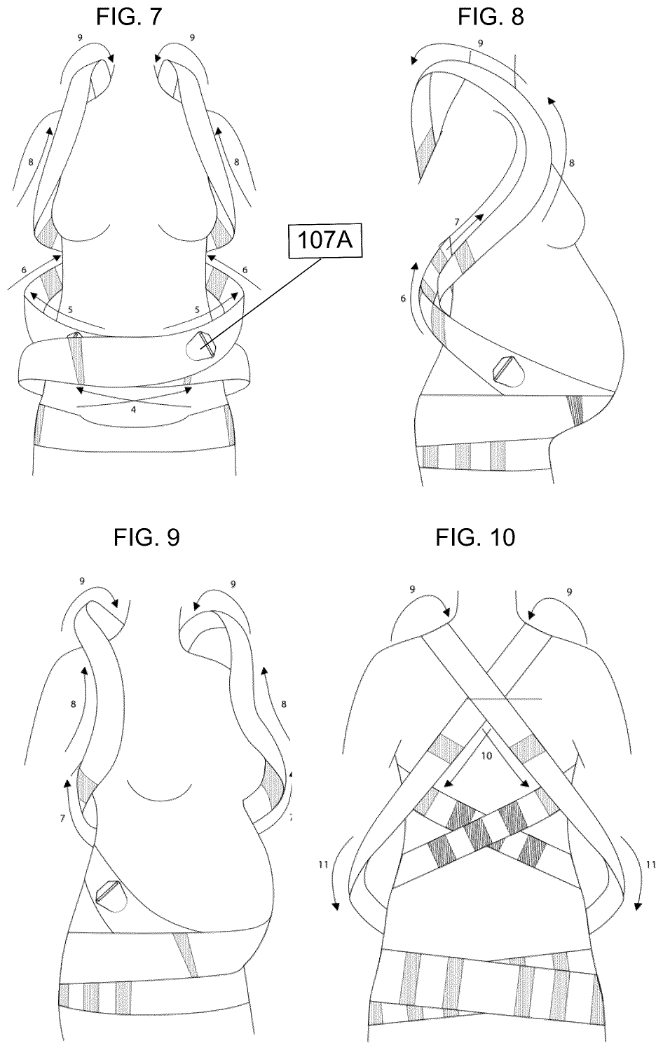

Referring now to FIGS. 3 and 4, the sling 100 symmetrically wraps over the iliac crest of the pregnant wearer and forms a first criss-cross 105 in the hip region. Referring to FIGS. 5 and 6, the sling 100 is subsequently wrapped around the axillary region of the pregnant wearer before making a second criss-cross pattern 106 in the upper lumbar/lower thoracic region. The second criss-cross pattern 106 is located above the first criss-cross 105. Referring to FIGS. 7-9, each end of sling 100 is draped over the shoulders. Referring to FIGS. 10-14, the sling 100 is positioned diagonally along the body's axis, culminating in a secure attachment to an alternate section of the sling 100 using a designated fastening point equipped with a buckle 130.

The weight and thus the size of the growing abdomen fluctuates during pregnancy and changes rapidly during the last months. In some cases, the shape and weight of the abdomen also change for some people experiencing swelling throughout the day. As such, there is a need for a personalized and easy-to-use fastening system. The embodiment of the garment illustrated at FIGS. 2-14 comprises a plurality of fastening points 107A or 107B. In such embodiments, the garment comprises at least one fastening point 107 on each side. The fastening points 107A or 107B are strategically positioned to offer enhanced adjustability and a customizable fit. As such, the sling 100 generally aims at being finely tuned to accommodate and support the wearer's back, hip and pelvic girdle more effectively. The fastening points generally facilitates adjustability, aiming to ensure a precision fit, to conform to the wearer's body, and to provide targeted stabilization to the hip and pelvic areas, which is crucial for promoting proper posture and alleviating discomfort during pregnancy. In other embodiments, the sling 100 comprises alternate fastening points 107A and 107B.

Referring now to FIGS. 6 and 7, embodiments of the garment comprising different fastening points 107A are illustrated. In such embodiments, the garment comprises two fastening points 107A for adjusting the sling 100. The fastening points 106 generally aim at fitting and stabilizing the hip and pelvic girdle. The first fastening point 107 adjusts the sling in the upper portion of the torso. The second fastening point 108 allows for tightening of the lower part of the sling.

Referring now to FIGS. 7 and 8, a sling 100 made of material having different properties at different locations is illustrated. The sling 100 The sling 100 comprises flexible, stretchable panels 102 combined with portions 103 made of a more rigid and supportive material. The rigid portions 103 have particular use in supporting and stabilizing the pelvic girdle from below and upward. The elastic panels 102 are seamlessly inserted in the belly support sling 100 to allow the sling 100 to fit fully around the abdomen and better disperse or distribute weight by transferring the said weight to other parts of the body not involved in belly carrying. The elastic panels 102 may further provide a smoothing effect. The elasticity in the thoracic spine promotes better adjustment and comfort for the user, while the support provided by rigid material panels actively reduces kyphosis.

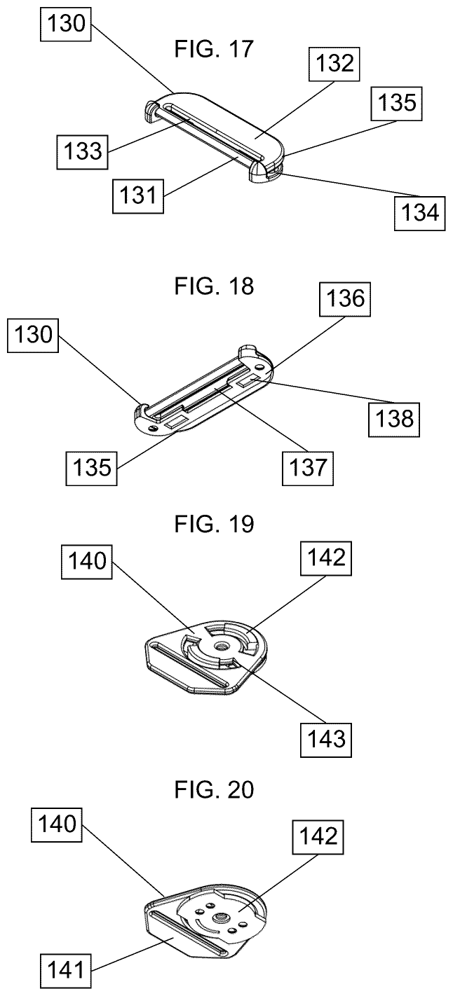

Referring now to FIGS. 17 to 20, two embodiments of fastening systems 125 are illustrated. Referring to FIGS. 17 and 18, the illustrated fastening system 125 is a male/female-type detachable buckle 130 interconnecting the sling straps 100. The elongated buckle 130 shown in FIGS. 17 and 18 includes a channel or recess 131. The central channel 131 is formed by a central bar 132 extending along the width of the fastening system. The channel 131 is adapted to receive a threaded end 133 of a strap. One end 134 is equipped with a locking mechanism 135, providing the means to secure or release the strap. The other end 136 is designed to anchor the sling's end firmly. The top end 134, illustrated at FIG. 17, ends in a clasp that, when inserted, perfectly connects with the aperture or hole 137 of the bottom part 136, illustrated at FIG. 18 of the buckle 130 and locks the buckle 130 in place with the use of magnets 138.

Referring now to FIGS. 19 and 20, another embodiment of the fastening system 125 is illustrated. The second embodiment comprises a round rotational buckle 140. The round rotational buckle 140 comprises a main body 141 having a central circular hub 142 adapted for rotational function and a guide 143 radiating from the circular hub. The guide 143 is adapted to align a strap and to secure the said strap in place. The rotation feature generally ensures optimal alignment while allowing for a slight strap movement, and the quick-release buckle 140 facilitates effortless placement of the sling. Understandably, any known fastening system 125 allowing locking the sling 100 in place is within the scope the present invention.

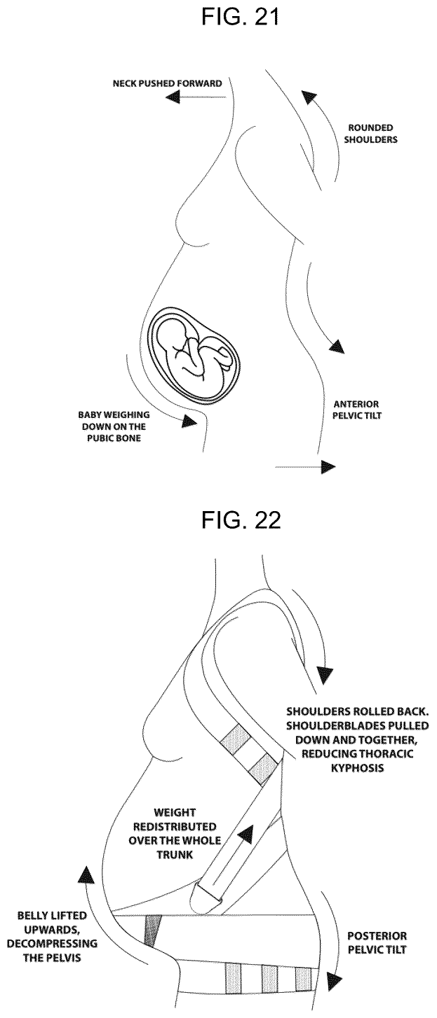

Referring now to FIG. 21, the forces created by the weight of the growing fetus on a pregnant woman are illustrated. Downward forces are generated on the pubic bone. The anterior positioning of the load leads to biomechanical changes including anterior pelvic tilt, thoracic kyphosis and a forward head posture. Such changes in posture are often associated with pain and discomfort due to the increased stress on the spine. As shown on the FIG. 22, the biomechanical effects of the sling 100 lead to a decompression of the pelvic region and added support of the belly. The force vector illustrates the transfer of the load onto the torso. This load transfer encourages a posterior pelvic tilt decompressing the pelvic spine. The sling 100 also transfers the load onto the shoulders, leading to a neutral thoracic spine and shoulder position.

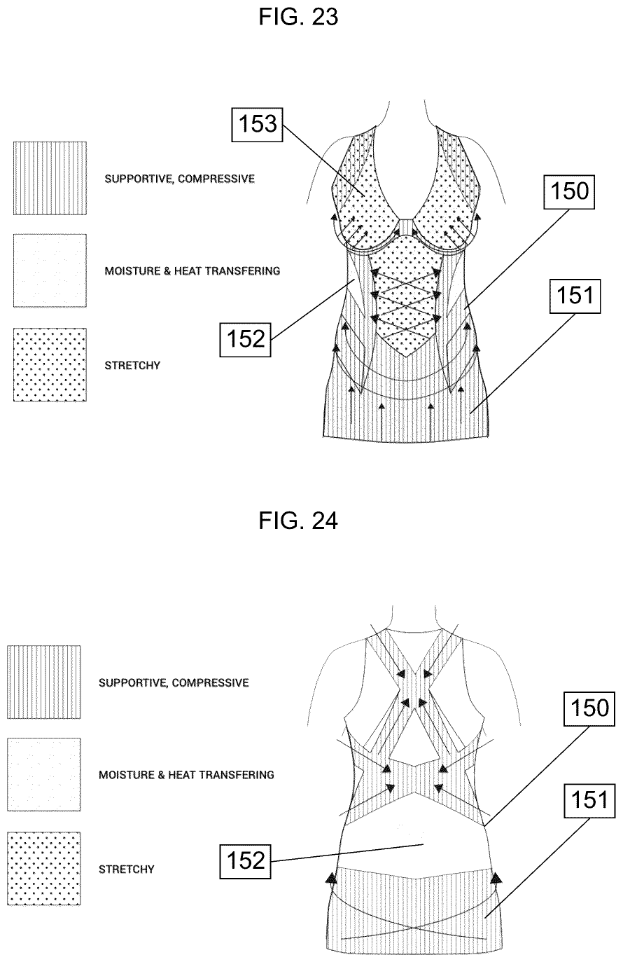

Referring now to FIGS. 23 and 24, the different properties in the material of a supportive bodysuit 150 in accordance with the principles of the present invention are shown. In such embodiment, the bodysuit 150 is made using 3D knitting. The 3D knitting process and the different materials of the bodysuit 150 generally creates four distinctive properties in the bodysuit, such as supportive/compressive 151, heat transfer and moisture wicking 152 and stretchy 153. The supportive and compressive fibers 151 follow the path of the sling 100 as previously described to support the fetus and to distribute the load onto the shoulder and trunk of the pregnant wearer. The heat transfer properties 152 are concentrated in the thoracic region, an important source of heat generation in the human body. The majority of the bodysuit 150 presents breathable fabric and moisture wicking capability. Lastly, the material located in the breast and abdominal region presents more flexible properties 153. The flexible properties 153 can be obtained using a single yarn, such as employed in a 3D knitting technique that utilizes varied patterns and structures within the weave.

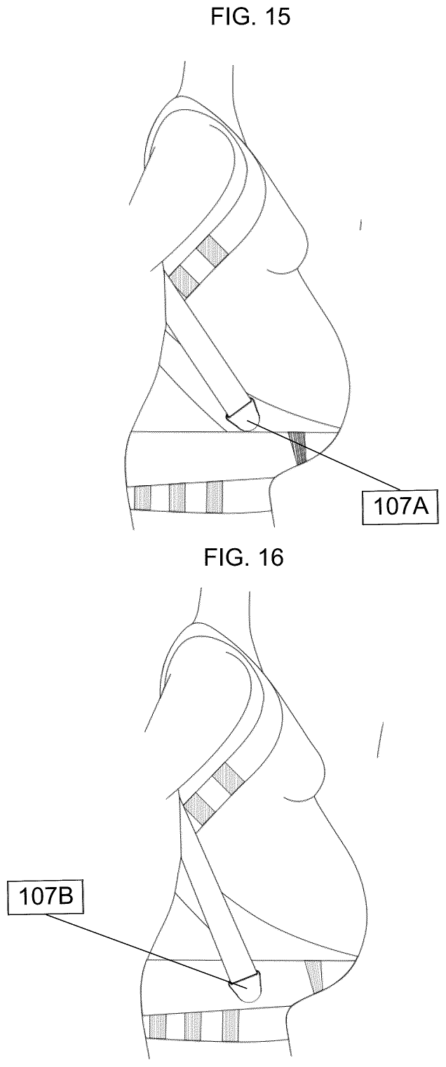

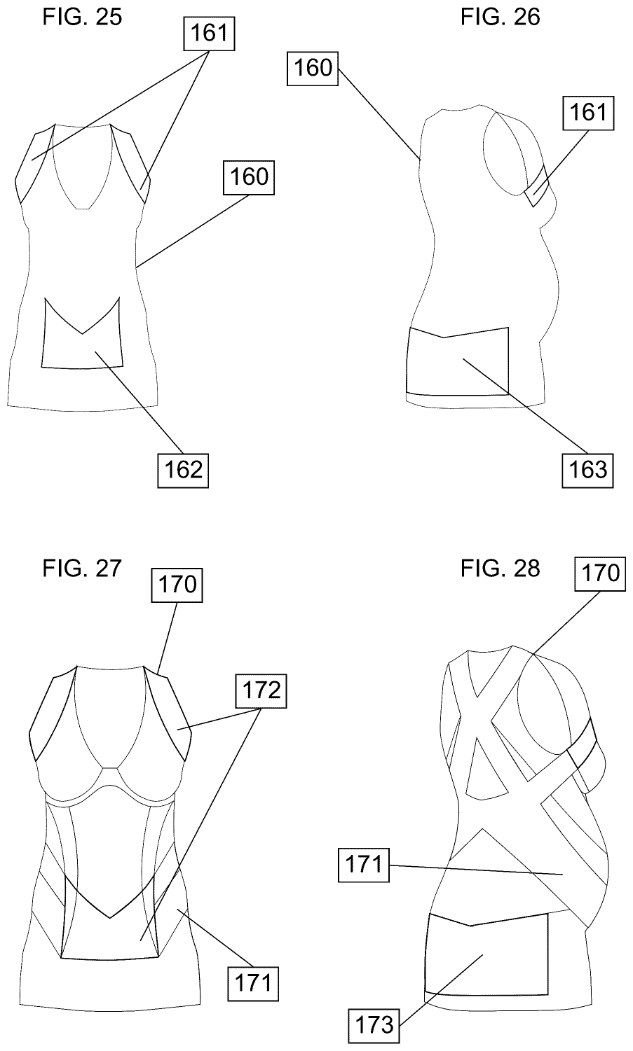

Referring now to FIGS. 25 and 26, a simplified embodiment of a top garment 160 in which a sling is assembled is illustrated. As illustrated, placement of the tunnels 161, 162 or 163 generally enables a symmetrical configuration in accordance with the principles of the present invention. The tunnels 161 to 163 generally allows the sling 100 to maintain a substantially constant pressure on the body of the wearer regardless of the position of the said wearer (ex: sitting, standing, etc.). The tunnels 161 to 163 also allow the sling 100 to be positioned by default on the user according to FIGS. 2 to 16. Having a preassembled sling 100 in the garment 160 further ensure easy or fast anatomic installation of the sling 100 when putting on the said garment.

FIGS. 25 and 26 further depict the top garment 160 in a ready for use state. As such, the interaction between the sling 100 and the top components are highlighted. the garment 160 comprises two top tunnels 161 positioned around the arms. The top garment 160 further comprises a bottom tunnel 162 located over a lower part of the abdomen. The said garment 160 may also comprise a back tunnel 163 aligned with the pelvic portion of the garment and situated near the sacrum.

Referring now to FIGS. 27 and 28, an embodiment of a bodysuit 170 is illustrated. The bodysuit 170 comprises a plurality of knitted structures 171 shown prior to the assembly or insertion of the sling 100. The bodysuit 170 comprises sewn-on portions of fabric at the front 172 and back 173. Such sewn-on portions 172 or 173 create tunnels in which the sling may be concealed. Such concealment generally aims at easing the donning for the user of the bodysuit and at facilitating correction of anatomical positioning of the sling 100.

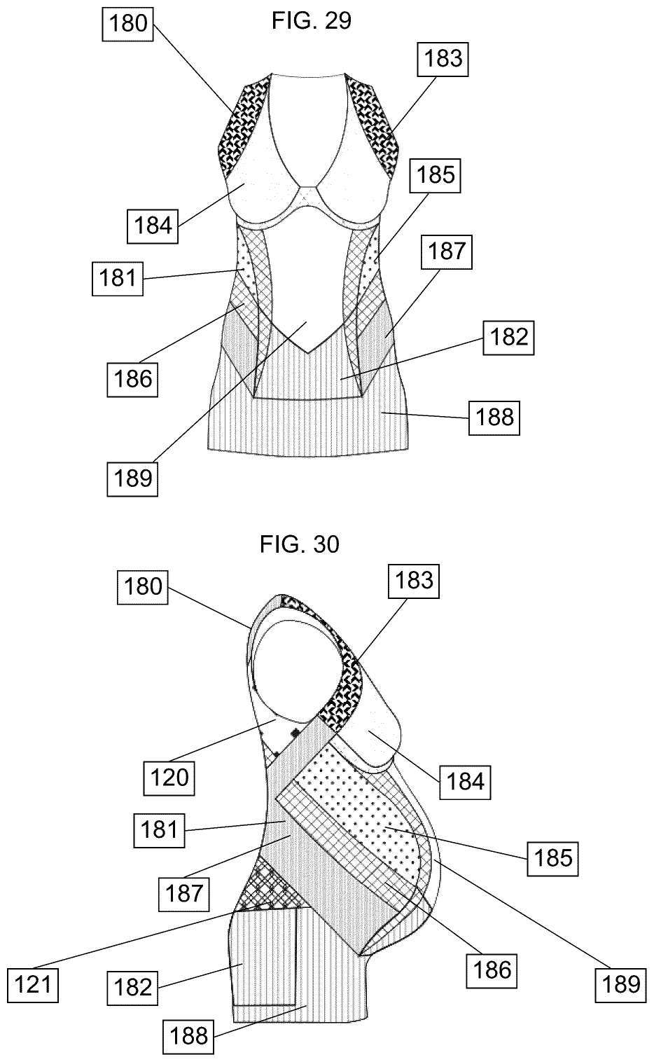

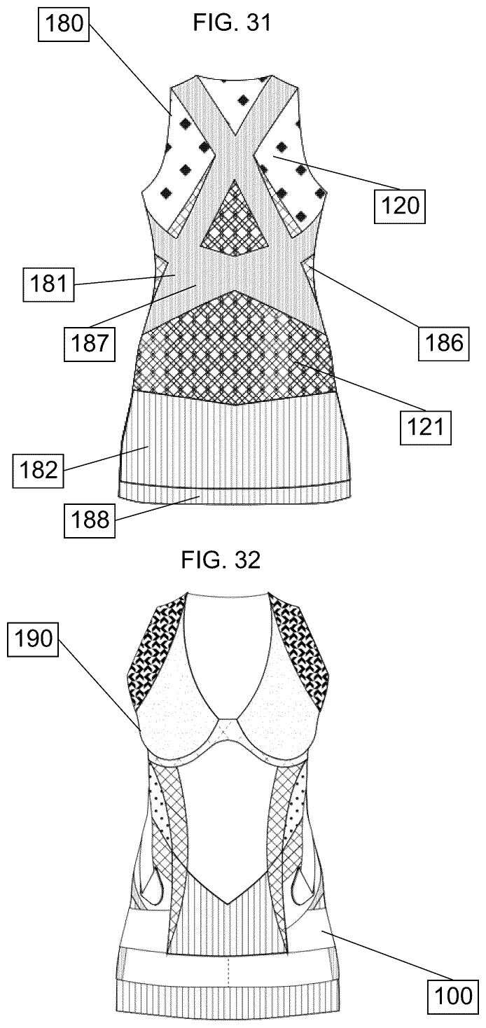

Referring to FIGS. 29 to 31, an embodiment of a top garment 180 comprising a plurality of structures 181 and tunnels 182 that are knitted together in the said top garment 180 are illustrated. The top garment 180 comprises distinct functional zones 183, 184, 185, 186, 187, 188 and/or 189 aiming at ensuring an optimal fit and enhanced sling functionality for the user. A top zone 183 comprises knitted fabric embroidered in the shoulder area. A bust zone 184 located in the bust area comprises knitted fabric having more stretch and less thickness than other zones. Lateral zones 185, 186, and 187 comprise differentiated diagonal structures adapted to provide abdominal support and conform to the sling's contour. A lower zone 188 comprises a ribbed texture being more rigid than other zones. The lower zone 188 generally delivers increased support and stabilizes the suit's position. A central zone 189 is made of stretching material, similarly to the top zone 183. The central zone 189 is generally adapted to accommodate extensive stretching to adapt to the abdominal expansion during pregnancy. The garment 180 further comprise moisture and/or heat transfer sections 120 and 121. Such sections 120 and 121 are made of breathable and elastic material. Preferably, the material of sections 120 and 121 have an elasticity level which is lower than the top zone 183 and central zone 189.

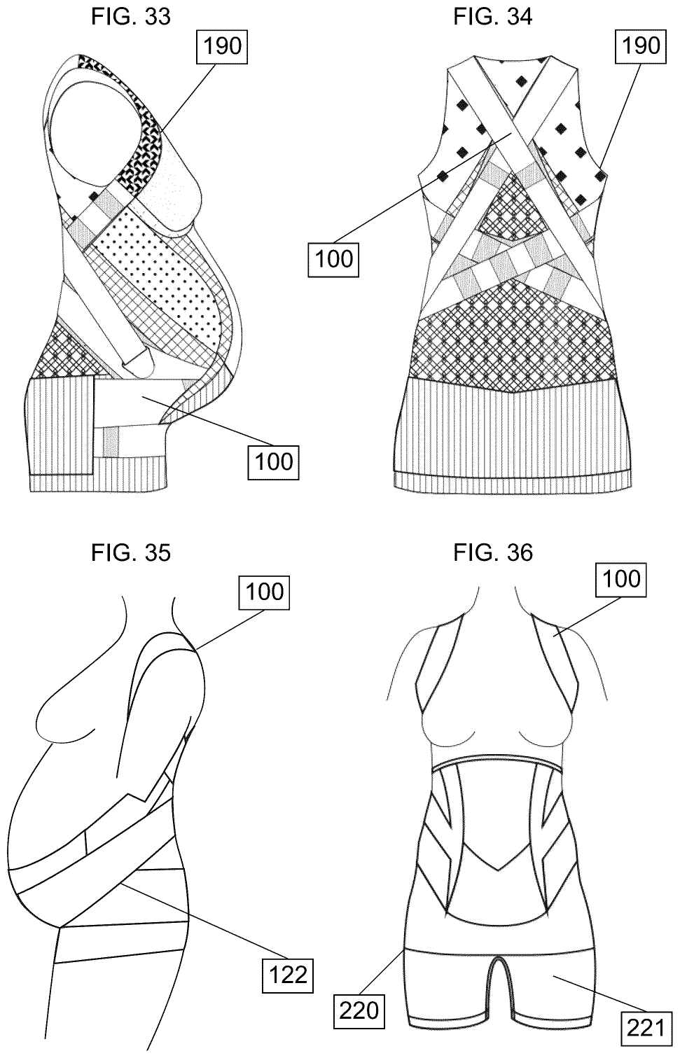

Referring to FIGS. 32 to 34, an embodiment of the main body of the sling 100 is illustrated. In such an embodiment, the sling 100 is assembled within a bodysuit 190 having various structures, such as the zones illustrated at FIGS. 29 to 31. As illustrated, the sling 100 is positioned on the exterior of the bodysuit 190. As such, the contribution of each of the varying structures or zones to the sling's enhanced functionality is illustrated.

Referring now to FIG. 35, another embodiment of the sling 100 is illustrated. The sling 100 further comprises an additional strap 122. The additional strap 122 is installed on the pregnant wearer to further support the weight of the belly. The additional strap 122 is wrapped from anterior to posterior.

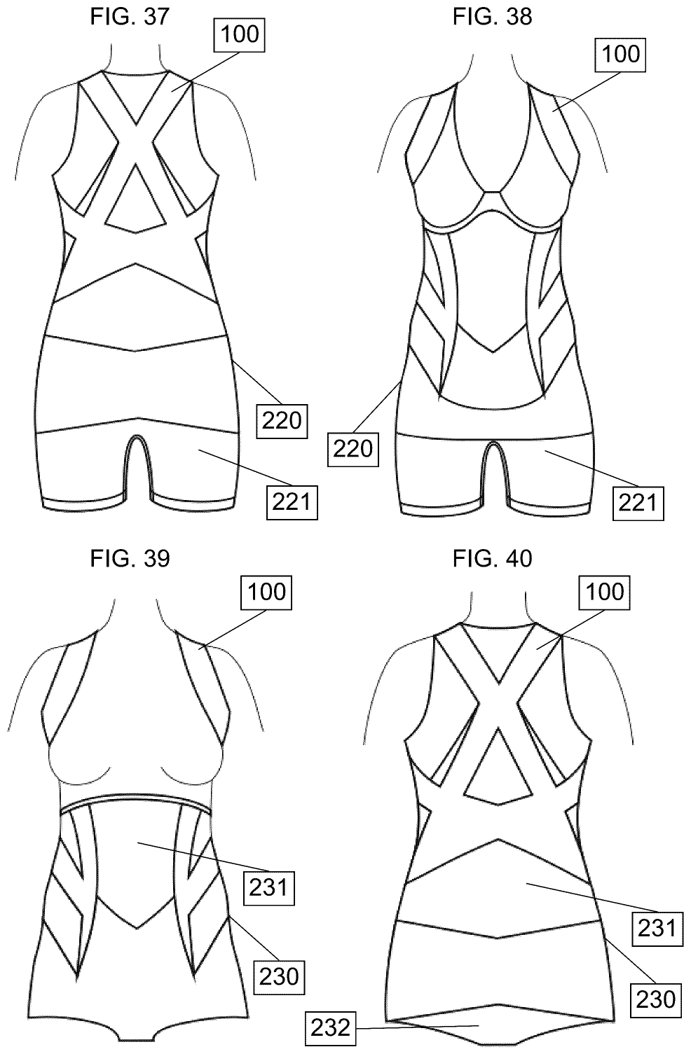

Referring now to FIGS. 36 to 43, different embodiments 220, 230, 240 or 250 of a sling 100 embedded in a bodysuit or garment are illustrated. In such embodiments, the device comprises a sling that serves as the basis for the construction that is embedded into one of the embodiments of a garment or undergarment. The sling 100 generally stabilizes the core and pelvis, while actively dispersing and/or distributing the growing abdomen's weight over different parts of the body and reducing internal organ pressure with a fully customizable level of support. The outer portion generally acts as a shell, allowing for easier wearing and taking off of the garment. The outer portion may further improve the silhouette of the pregnant wearer. As such, in such embodiments, the garment 220, 230, 240 or 250 may have different configurations, such as comprising briefs, shorts or built-in underwear.

The use of 3D-knitting technology allows having a plurality of different material properties in one garment. The sling may be made of materials having different properties, such as compression/elastic material and semi-rigid materials in the lower portion of the garment. The materials having different properties generally stabilize the pelvis as the outer portion of the material enhances the ability of the sling 100 to stabilize the pelvis. Based on the knowledge gathered from research on anatomy, thermoregulation, women's specific sweat and heat zones, as well as areas of greatest stress, the right knit fabric was selected having specific properties where they were most desired, while also providing optimal functionality for maximum comfort while wearing the garment, including but not limited to moisture-wicking, heat transferring and loose stretchy zones. Understandably, any type of fabric known in the art having the above-mentioned properties may be used within the scope of the present invention.

Referring now to FIGS. 36-38, an embodiment of a one-piece bodysuit 220 comprising a built-in sling and built-in shorts 221 is illustrated. As some qualitative research suggests, plus-size pregnant people might prefer shorts, pants or leggings to girdles as a type of support garment. To provide an embodiment that would answer the needs of such wearers, the sling 100 is placed within a one-piece bodysuit with shorts 221. It might also be a preferred embodiment for users with excessive leg swelling counteracting rubbing and irritation of the thigh skin. The garment of the present embodiment generally uses the same technology and properties of the sling 100, but with an additional portion of 3D-knitted fabric, it might provide more satisfactory posture correction and holistic support within the lumbar and chest area.

Referring to FIGS. 39 and 40, another embodiment of a garment 230 comprising a pre-assembled sling 100 is illustrated. In such an embodiment, the sling 100 is attached to additional panels 231 located in the abdominal region of the pregnant wearer. The sling 100 is further attached to a built-in underwear 232 component to further support the most vulnerable area affected by abdominal weight. The additional panels 231 and/or underwear 232 component generally aim at enhancing the perceived level of comfort while a user wears the garment. The additional panels 231 and/or underwear 232 component are preferably knitted to the sling 100. The said panels 231 and underwear component 232 act as an extension of the sling 100 and may help counteract the lordotic effect by stabilizing the pelvis and coccyx bones. The garment 230 generally comprises a variety of compression levels which may also support and transfer/distribute weight of the abdomen of the wearer more efficiently. Furthermore, by having the sling 100 built into the underpants 232, putting on and taking off the sling 100 is significantly easier and faster for the wearer.

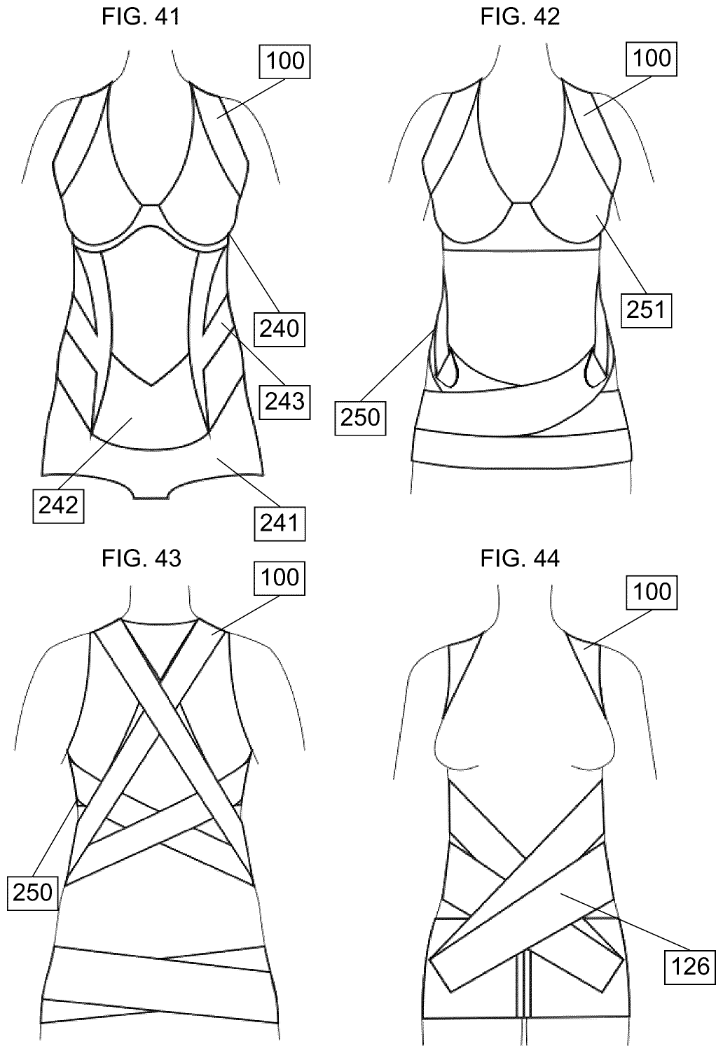

FIG. 41 illustrates an embodiment of a one-piece bodysuit 240 including an underwear portion 241 and a built-in sling 100. The bodysuit 240 with a built-in sling 100 generally aims at casing putting on and taking off the sling. The back of the bodysuit is very similar to the embodiment of the garment 230 shown at FIG. 40. The embedded sling 100 within the bodysuit 240 generally allows for an effective utilization of 3D knitting technology. In embodiments made with 3D knitting technology, a continuous piece of fabric may comprise a plurality of portions 242 and 243. Each portion 242 and 243 may have specific elastic properties to create a desired effect in the sling. Some of the effects comprise overall support, pelvis stabilization and abdominal weight redistribution. As an example, the fabric of the support areas may have semi-rigid or semi-elastic properties while the fabric under the abdomen and the bust may have more compressive or elastic properties. Such variations in clastic properties of the fabric generally contribute to improve correction of the posture of a wearer by stabilizing joints and counteracting lordosis and kyphosis, particularly in wearers having large breasts and wide cleavage.

Referring now to FIGS. 42 and 43, an embodiment of a one-piece sling 100 assembly is illustrated. In such an embodiment, the sling 100 is pre-assembled into a wearable shape. This pre-assembled design facilitates the process of putting on the sling 100. Indeed, some research indicates that a complex design of support garments may negatively influence the perceived safety of the garment by the users. In such an embodiment, the garment 250 may comprise an optional built-in bra 251 is illustrated. Similar to the embodiment shown in FIG. 29, the different levels of thickness and compression of the knitted fabrics present in the garment 250 generally help transferring or distributing the weight more efficiently. Therefore, the built-in bra 251 may provide additional support and may improve posture for people with larger breasts and cleavage areas who are prone to kyphosis. The garment 250 may also be used as prophylactic wear of the sling 100 by the wearer. As such, the garment 250 may be used as a total support device without the need to wear additional underwear.

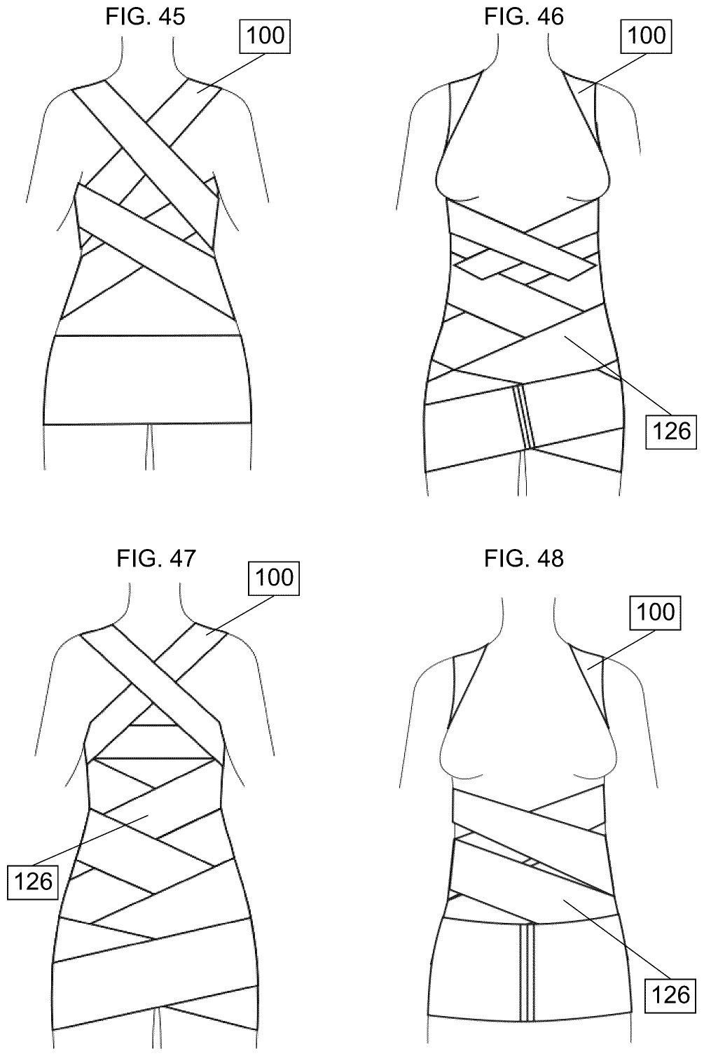

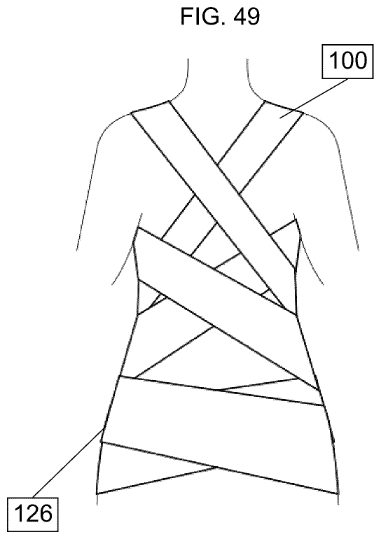

Referring now to FIGS. 44 to 49, a plurality of embodiments of the sling 100 are illustrated. Such embodiments are generally designed for multipurpose functionality, including general-use support and targeted postpartum and surgical recovery compression. In such embodiments, the sling 100 aims at facilitating an improved posture and delivering robust back support. Distinctively, these embodiments incorporate a specialized front belly portion 126, meticulously crafted to provide additional support and foster healing post-surgery and post-childbirth. This front belly portion 126, when worn, generally aims at applying gentle yet effective compression, aiding in the stabilization of the abdominal region while conforming to the body's natural contours to ensure comfort during the recovery process. As such, the front belly portion 126 does not constrict around the entire waist of the wearer thus ensuring improved mobility of the abdomen compared to methods constricting the entire or a substantial portion of the waist.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

1. A continuous body sling for supporting and distributing the load of the abdomen of a wearer, the body sling comprising:
a pelvic portion positioned to support the belly of the wearer and to stabilize the pelvis of the wearer;
a shoulder portion adapted to wrap around the shoulders of the wearer to transfer at least some of the load onto the shoulders and neck;
at least one crossing of the sling in the back of the wearer to distribute some of the load onto the trunk; and
a fastening mechanism adapted to vary the length of the worn sling at a plurality of fastening points;
wherein at least one of the portions is made of elastic material to accommodate changing contours of a wearer's body.

2. The body sling of claim 1, the pelvic portion being anatomically shaped to fit the body of the wearer and positioned at a first position in the suprapubic or hypogastric region supporting the weight of the belly of a wearer.

3. The body sling of claim 1 forming two crossings in the back of the wearer.

4. The body sling of claim 1 forming three crossings in the back of the wearer.

5. The body sling of claim 1 being made of 3D knitted fabric.

6. The body sling of claim 1 being made with moisture wicking and breathable material.

7. The body sling of claim 1 being made with heat transferring material.

8. The body sling of claim 1 being made with material alternating between material having different elastic properties.

9. The body sling of claim 1 being made with material alternating between elastic and non-elastic material.

10. The body sling of claim 9, wherein the series of elastic sections provided in the sling are interspersed with non-elastic sections, wherein the non-elastic sections are integrated to furnish consistent support, ensuring that the sling retains its structural integrity while in use.

11. The body sling of claim 1 being made with material alternating between elastic and semi-rigid material.

12. The body sling of claim 1, wherein the material has higher rigidity in the pelvic portion.

13. The body sling of claim 1, wherein the material of the sling is of more supple in portions of the sling to be worn on the axillary region of the wearer than in other portions of the sling.

14. The body sling of claim 1, the fastening mechanism comprising a detachable buckle allowing adjustment of strap length to personal setting.

15. The body sling of claim 14, the detachable buckle comprising multiple tension settings.

16. The body sling of claim 1 comprising multiple shoulder portions to improve load distribution.

17. The body sling of claim 1 being adaptable to the weight of the abdomen being increased by a growing foetus, wherein the wearer is a pregnant woman.

18. The body sling of claim 1, the weight of the abdomen being increased by a growing foetus and the wearer being a pregnant woman.

19. A bodysuit comprising tunnels for allowing passage of a built-in body sling for supporting and distributing the load of the abdomen of a wearer throughout the body trunk of the said wearer, the body sling comprising:
shoulder portions to transfer at least some of the load onto the shoulders and neck;
a pelvic portion positioned between the shoulder portions to support the belly of the wearer and stabilizing the pelvis of the wearer; and
at least one crossing of the sling in the back of the wearer to distribute some of the load onto the trunk of the wearer;
wherein at least one of the portions of the body sling is made of elastic material to accommodate changing contours of a wearer's body.

20. The bodysuit of claim 19 being made of moisture wicking, heat transferring and breathable material.

21. The bodysuit of claim 19 being 3D knitted.

22. The bodysuit of claim 19, the built-in body sling forming two crossings in the back of the wearer.

23. The bodysuit of claim 22, the built-in body sling forming three crossings in the back of the wearer.

24. The bodysuit of claim 19, the series of elastic sections of the sling being interspersed with non-elastic sections, wherein the non-elastic sections are integrated to furnish consistent support, ensuring that the sling retains its structural integrity while in use.

25. The bodysuit of claim 19 being a maternity bodysuit.

26. The bodysuit of claim 19 being adaptable to the weight of the abdomen being increased by a growing foetus, wherein the wearer is a pregnant woman.

27. A method to support and distribute weight of the abdomen of a wearer throughout the body trunk of the said wearer, the method comprising:
supporting a pelvic portion of the belly of the wearer using a continuous sling;
wrap the continuous sling around the shoulder and the trunk of the wearer;
crossing the continuous sling in the back of wearer; and
attaching a first end of the sling to a second end of the sling to provide a sufficient compression level on the wearer.

28. The method of claim 27 further comprising crossing the continuous sling a second time in the back of the wearer.
